SEQUENCE LISTING (48 sequences)

SEQ ID NO: 1 - DNA, 20 bp, Artificial
  oligonucleotide primer
cctctgaaagaggaacttgg

SEQ ID NO: 2 - DNA, 59 bp, Artificial
  oligonucleotide primer
caacaattgcattcattggcgcgccgcggccgcttaattaactttgcaaagctttttgc

SEQ ID NO: 3 - DNA, 59 bp, Artificial
  oligonucleotide primer
gcaaaaagctttgcaaagttaattaagcggccgcggcgcgccaatgaatgcaattgttg

SEQ ID NO: 4 - DNA, 32 bp, Artificial
  oligonucleotide primer
cacagaggagcttcctggggatccggtaccag

SEQ ID NO: 5 - DNA, 28 bp, Artificial
  oligonucleotide primer
cgggatccagacatgataagatacattg

SEQ ID NO: 6 - DNA, 42 bp, Artificial
  oligonucleotide primer
atagtttagcggccgcaacttgtttattgcagcttataatgg

SEQ ID NO: 7 - DNA, 30 bp, Artificial
  oligonucleotide primer
cttaattaagggtctctctggttagaccag

SEQ ID NO: 8 - DNA, 31 bp, Artificial
  oligonucleotide primer
agcggccgcagtcgcctcccctcgcctcttg

SEQ ID NO: 9 - DNA, 29 bp, Artificial
  oligonucleotide primer
caggtgtcgtgagtagcaccatccaaagg

SEQ ID NO: 10 - DNA, 40 bp, Artificial
  oligonucleotide primer
atagtttagcggccgcacaagtagcaatacagcagctacc

SEQ ID NO: 11 - DNA, 31 bp, Artificial
  oligonucleotide primer
agcctaggacaagtagcaatacagcagctac

SEQ ID NO: 12 - DNA, 30 bp, Artificial
  oligonucleotide primer
agaaggcacaggtagcaccatccaaaggtc

SEQ ID NO: 13 - DNA, 28 bp, Artificial
  oligonucleotide primer
gcttaattaaccagcggaaagtcccttg

SEQ ID NO: 14 - DNA, 28 bp, Artificial
  oligonucleotide primer
gagcggccgccactttgtacaagaaagc

SEQ ID NO: 15 - DNA, 28 bp, Artificial
  oligonucleotide primer
gagcggccgcacagatccattcgattag

SEQ ID NO: 16 - DNA, 26 bp, Artificial
  oligonucleotide primer
gaggcgcgccagcggaaagtcccttg

SEQ ID NO: 17 - DNA, 30 bp, Artificial
  oligonucleotide primer
gcttaattaaggttggggagtagatagatg

SEQ ID NO: 18 - DNA, 28 bp, Artificial
  oligonucleotide primer
gagcggccgccgtgttgaggagtcaatc

SEQ ID NO: 19 - DNA, 28 bp, Artificial
  oligonucleotide primer
gagcggccgcgagcggctttacatcggg

SEQ ID NO: 20 - DNA, 30 bp, Artificial
  oligonucleotide primer
gaggcgcgccactgtgctggatatcaaacc

SEQ ID NO: 21 - DNA, 30 bp, Artificial
  oligonucleotide primer
cggcgcgccgggtctctctggttagaccag

SEQ ID NO: 22 - DNA, 31 bp, Artificial
  oligonucleotide primer
agtttaaacagtcgcctcccctcgcctcttg

SEQ ID NO: 23 - DNA, 29 bp, Artificial
  oligonucleotide primer
cgtttaaactgagtagcaccatccaaagg

SEQ ID NO: 24 - DNA, 28 bp, Artificial
  oligonucleotide primer
gagtttaaaccactttgtacaagaaagc

SEQ ID NO: 25 - DNA, 33 bp, Artificial
  oligonucleotide primer
agcctgcaggacaagtagcaatacagcagctac

SEQ ID NO: 26 - DNA, 30 bp, Artificial
  oligonucleotide primer
attaattaaaggtagcaccatccaaaggtc

SEQ ID NO: 27 - DNA, 28 bp, Artificial
  oligonucleotide primer
gcggcgcgccccagcggaaagtcccttg

SEQ ID NO: 28 - DNA, 28 bp, Artificial
  oligonucleotide primer
gagtttaaaccactttgtacaagaaagc

SEQ ID NO: 29 - DNA, 28 bp, Artificial
  oligonucleotide primer
gagtttaaacacagatccattcgattag

SEQ ID NO: 30 - DNA, 26 bp, Artificial
  oligonucleotide primer
gattaattaaagcggaaagtcccttg

SEQ ID NO: 31 - DNA, 30 bp, Artificial
  oligonucleotide primer
gcggcgcgccggttggggagtagatagatg

SEQ ID NO: 32 - DNA, 28 bp, Artificial
  oligonucleotide primer
gagtttaaaccgtgttgaggagtcaatc

SEQ ID NO: 33 - DNA, 28 bp, Artificial
  oligonucleotide primer
gagtttaaacgagcggctttacatcggg

SEQ ID NO: 34 - DNA, 30 bp, Artificial
  oligonucleotide primer
gattaattaaactgtgctggatatcaaacc

SEQ ID NO: 35 - DNA, 26 bp, Artificial
  oligonucleotide primer
gcgaattcgtgaggctccggtgcccg

SEQ ID NO: 36 - DNA, 38 bp, Artificial
  oligonucleotide primer
caatatagatcttagacatgcacgacacctgaaatgga

SEQ ID NO: 37 - DNA, 38 bp, Artificial
  oligonucleotide primer
tccatttcaggtgtcgtgcatgtctaagatctatattg

SEQ ID NO: 38 - DNA, 39 bp, Artificial
  oligonucleotide primer
tggcaactagaaggcacagctaatgatgacgtaaccaag

SEQ ID NO: 39 - DNA, 39 bp, Artificial
  oligonucleotide primer
cttggttacgtcatcattagctgtgccttctagttgcca

SEQ ID NO: 40 - DNA, 375 bp, Artificial
  oligonucleotide primer
gcgaattccatagagccccgcatccccvdvdnaartcagcggatccccggaagaaatatatttgcvdvdnaartcagcggatccgctatggcagggcctgccgvdvdnaartcagcggtaccgtgaggctccggtgcccgvdvdnaartcatgtttaaaactttatccatgcacgacacctgaaatggavdvdnaartcatccatttcaggtgtcgtgcatggataaagttttaaacavdvdnaartcatggcaactagaaggcacagttatgtttcaggttcaggggvdvdnaartcacccctgaacctgaaacataactgtgccttctagttgccavdvdnaartcagcggtaccatagagccccgcatcccc

SEQ ID NO: 41 - DNA, 27 bp, Artificial
  oligonucleotide primer
gcggatccccggaagaaatatatttgc

SEQ ID NO: 42 - DNA, 27 bp, Artificial
  oligonucleotide primer
gcggatccgctatggcagggcctgccg

SEQ ID NO: 43 - DNA, 26 bp, Artificial
  oligonucleotide primer
gcggtaccgtgaggctccggtgcccg

SEQ ID NO: 44 - DNA, 38 bp, Artificial
  oligonucleotide primer
tgtttaaaactttatccatgcacgacacctgaaatgga

SEQ ID NO: 45 - DNA, 38 bp, Artificial
  oligonucleotide primer
tccatttcaggtgtcgtgcatggataaagttttaaaca

SEQ ID NO: 46 - DNA, 39 bp, Artificial
  oligonucleotide primer
tggcaactagaaggcacagttatgtttcaggttcagggg

SEQ ID NO: 47 - DNA, 39 bp, Artificial
  oligonucleotide primer
cccctgaacctgaaacataactgtgccttctagttgcca

SEQ ID NO: 48 - DNA, 26 bp, Artificial
  oligonucleotide primer
gcggtaccatagagccccgcatcccc

(19) United States
(12) Patent Application Publication
(10) Pub. No.: US 2010/0129913 A1
(43) Pub. Date: May 27, 2010

(54) POLIOMA VECTOR EXPRESSING LONG DOUBLE-STRANDED RNAS

(76) Inventors: Gerrit Jan Van Holst, Enkhuizen (NL); Petrus Theodorus De Haan, Oestgeest (NL)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 12/306,585

(22) PCT Filed: Jun. 27, 2007
(86) PCT No.: PCT/EP2007/056450

§ 371 Date: Feb. 4, 2010

(30) Foreign Application Priority Data
Jun. 27, 2006 (GB) 0612694.0

(51) Int. Cl. C12N 15/00; C12N 5/00; C12N 15/63; C07H 21/00

(52) U.S. Cl. 435/455; 435/325; 435/320.1; 536/23.1

(57) ABSTRACT

Polyoma viral vector production cell line comprising a heterologous polynucleotide sequence that is capable of being transcribed into an RNA sequence that is capable of folding into double stranded RNA of at least 50 base pairs in length, methods of producing said cell line, uses thereof and recombinant polyoma viral vectors and nucleic acid sequences relating thereto.

The present invention relates to the production of vaccines and therapies for the treatment and/or prophylaxis of viral disease. In particular, the invention relates to the production of viral vectors, plasmids carrying viral sequences, virus packaging cell lines such as polyoma virus wild type free packaging cell lines, methods of producing viral vectors, such as polyoma viral vectors e.g. SV40 viral vectors, host cells therefor, compositions comprising viral vectors and uses thereof, and vaccines against viruses such as HIV-1, HBV and HCV.

Polyoma viral vectors, including vectors derived from Simian virus 40 (SV40) are known as potential vectors for gene transfer into a plurality of human tissues, for example, bone marrow (Rund D. et al, Human Gene Therapy 9, 649-657 (1998) and the liver (Strayer D. S. and Zern M. A. Seminars in Liver Disease 19, 71-81 (1999). Polyoma viral vectors, such as SV40, are known to infect non-dividing as well as actively dividing cells and are also known to be non-immunogenic (Strayer supra (1999)) allowing repeated administration to the same individual. Moreover, it allows long-term expression of the transgene. As such, researchers have known for some time that the polyoma viral vectors, such as SV40-derived vectors, make promising candidates for therapeutic and/or prophylactic gene or nucleic acid transfer.

SV40 is an example of a non-enveloped polyoma virus belonging to the Papovaviridae with a 5.25 kilo base pair, long circular double stranded DNA genome. The SV40 genome consists of two regulatory regions, the promoter/origin region and the polyadenylation region. The promoter/origin region is 500 base pairs long and comprises two oppositely-directed promoters, the early and late promoter (SVEP and SVLP respectively) that flank the central origin of replication and packaging signal. The polyadenylation region is 100 base pairs long and contains the polyadenylation signals of both the early and the late transcripts. The early promoter drives expression of the small, medium and large T antigens (stag, mtag and Tag, respectively) necessary for virus replication and activation of the late promoter. The late promoter drives expression of the viral capsid proteins VP1, 2 and 3.

Certain problems associated with recombinant SV40 packaging cell lines, such as COS cell lines (Gluzman Y., Cell 23, 175-182 (1981) inter alia relate to the fact that wild type replication-competent SV40 particles are produced in the constructed packaging cell lines (Gluzman Y ibid; Oppenheim A., and Peleg A., Gene 77, 79-86,(1989)). Additionally, the presence of the replication competent wild type virus particles albeit in small amounts in such conventional packaging cell lines in the past has made the use of SV40 for medical purposes impractical.

WO 03/025189 describes an invention where it is alleged that the packaging complementation cell lines that are described therein allow for the production of SV40 viruses and so-called pseudo-viruses that are safe for medical use.

WO 03/025189 teaches that the problem of generating T-antigen-positive revertant viruses is eliminated in the engineered packaging cell lines of that invention. Thus the problem of generating recombinant viable viruses that carry and can express the T-antigen appears to be obviated.

While the invention of WO 03/025189 appears to provide a viable solution to the generation of safe SV40 viruses for use in medicine it appears that there is an additional problem highlighted in the art (Vera M., et al Molecular Therapy vol. 10, No. 4, October 2004) to do with the production of high titre stocks of suitable SV40 viral vectors. Vera M., et al show inter alia that the production capacity of recombinant SV40 viruses of interest in certain cell lines can be very low (e.g. in CMT4 and human embryonic kidney 293T (HEK293T)) and state that other cell lines, such as COT-2, are also not effective as producer cell lines for the recombinant SV40 virus particles utilised in that study.

It is an object of the present invention to provide more efficient packaging cell lines for recombinant polyoma virus particles, such as recombinant SV40 virus particles, that do not suffer from the disadvantages of packaging cell lines of the prior art.

It is a further object of the present invention to provide more reliable therapies against viral disease such as HIV-1-related disease and Hepatitis-related disease and others.

These and other objects of the invention will become apparent from the following description.

The present inventors have now found that certain viral nucleic acid sequences of a certain minimum length and encoding RNA molecules capable of folding into long double stranded RNA (dsRNA) molecules are surprisingly effective against cognate viruses and furthermore, that vectors, such as plasmids, comprising polyoma viral elements, such as SV40 viral elements, carrying the nucleic acid sequences encoding such virus-specific long dsRNA molecules that are used in the methods of production of recombinant SV40 virus particles in packaging cell lines give rise to better recombinant polyoma virus particles from such vectors or plasmids than those comprising nucleic acid sequences encoding shorter dsRNA sequences.

According to the present invention there is provided a polyoma viral vector production cell line comprising a heterologous polynucleotide sequence that is capable of being transcribed into an RNA sequence that is capable of folding into dsRNA of at least 50 base pairs in length, with nucleotide sequence having substantial homology to a target nucleic acid sequence of a vertebrate virus, the target nucleic acid sequence being an essential sequence of the said virus.

The term “nucleotide sequence homology” as used herein denotes the presence of homology between two polynucleotides. Polynucleotides have “homologous” sequences when either a sequence of nucleotides in the two polynucleotides is the same or when a sense sequence of the one and an antisense sequence of the other polynucleotide is the same when aligned for maximum correspondence. Sequence comparison between two or more polynucleotides is generally performed by comparing portions of at least two sequences over a comparison window to identify and compare local regions of sequence similarity. The comparison window is generally from about 20 to 200 contiguous nucleotides in length. The “percentage of sequence homology” for polynucleotide sequences of the invention, such as 50, 60, 70, 80, 90, 91, 92, 93, 94, 95, 96, 97, 98, 99 or 100 percent sequence homology may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul, S. F. et al. 1990. J. Mol. Biol. 215:403; Altschul, S. F. et al. 1997. Nucleic Acid Res. 25:3389-3402) and ClustalW programs both available on the internet. Other suitable programs include GAP, BESTFIT and FASTA in the Wisconsin Genetics Software Package (Genetics Computer Group (GCG), Madison, Wis., USA).

The homology between nucleic acid sequences may be determined with reference to the ability of the nucleic acid sequences to hybridise to each other upon denaturation (e.g., under conditions of 400 mM NaCl, 40 mM PIPES pH 6.4, 1 mM EDTA, at a temperature of 50° C. to 65° C. and hybridisation for 12-16 hours, followed by washing) (Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992).

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Sambrook et al 1989, supra.

The heterologous polynucleotide sequence(s) of the invention give rise to dsRNA molecules that in the target host cell are processed in the cytoplasm of the said host cell (mammalian, for example, human) by the dsRNA-specific endonuclease, “DICER”, into 19-26 nucleotides long small interfering RNAs (siRNAs) (Ketting R F et al. Genes Dev. 2001; 15: 2654-2659). These siRNAs are then incorporated into a multiprotein RNA-induced silencing complex (RISC) that guides the recognition and cleavage or translational repression of complementary single stranded RNA (ssRNA), such as messenger RNA or viral genomic RNA, causing inhibition of viral replication (Haasnoot P C J et al. J. Biomed. Sc. 2003; 10: 607-616). As such, it is important that the heterologous polynucleotide sequence(s) of the invention has(ve) a high sequence homology with the target sequence of the virus of interest, preferably at least 90% homology, more preferably 95% homology and even more preferably 98% or higher homology therewith.

The polyoma viral vector production cell line may be any one known in the art but is preferably selected from the VERO cell line (ref African Green Monkey kidney cell line ECACC 88020401, European Collection of Cell Cultures, Salisbury, Wiltshire, UK), the HEK293 cell line (ref Graham F L et al. J. gen. Virol. 1977; 36: 59-72), or the HEK293T cell line (ref DuBridge R B et al. Mol. Cell. Biol. 1987; 7: 379-387; and Rio D et al. Science 1985; 277: 23-28).

The choice of polyoma viral vector production cell line for the production of recombinant polyoma virus particles for use in therapy and/or prophylaxis requires that the production cell line has certain genetic elements comprised in it. Depending on the production cell line used, the T antigen genes (stag, mtag and Tag respectively) are either introduced into it or may already be present in the chromosomal DNA. The production cell line should preferably also comprise an RNA silencing suppressor and for use in antiviral therapy, a heterologous polynucleotide sequence of the invention as defined herein.

The polyoma viral vector production cell line is typically transfected with one or more DNA vectors carrying desired genetic components, such as plasmids, that may comprise one or more of the following components:

i) an RNA silencing suppressor sequence such as E3L from vaccinia virus, NS1 from Influenza virus A, VP35 from Ebola virus or Tat from HIV-1.

ii) a selectable marker such as a neomycin resistance gene, puromycin resistance gene, hygromycin resistance gene or other marker. Where the cell line in a preferment, includes an RNA silencing suppressor sequence stably integrated into the chromosomal DNA of the cell line, it may be further selected on the basis of the activity of the RNA silencing suppressor. Such markers and such selection procedures are well known in the art;

iii) A heterologous polynucleotide sequence of the invention as defined herein that is capable of being transcribed into an RNA sequence of at least 600 nucleotides long, with nucleotide sequence homology to the nucleic acid sequence of a vertebrate virus, that is capable of folding into dsRNA of at least 50 base pairs in length;

iv) an appropriate promoter such as the native SV40 early promoter (SVEP), or an heterologous promoter such as the cytomegalovirus immediate early promoter (CMVie) and the like;

v) an appropriate terminator such as the native SV40 polyadenylation sequence.

The term “heterologous” is used broadly throughout to indicate that the nucleic acid sequence, polynucleotide sequence, gene or sequence of nucleotides in question have been introduced into said polyoma viral vector producer cell line, using genetic engineering, i.e. by human intervention. A heterologous gene may in principle replace an endogenous equivalent gene, or be additional to the endogenous genes of the genome of the host cell or polyoma virus i.e. is non-naturally occurring in cells of the host species or in polyoma viruses.

The polynucleotide sequence of the invention is capable of being transcribed into a dsRNA or RNA duplex. Suitably therefore, the RNA comprises inverted repeats, or is of a palindromic structure but preferably comprises antisense and sense sequences. The dsRNA may be in the form of a hairpin structure or panhandle folding, whereby the sense and antisense regions are separated by introns or lariat structures that are removed during mRNA formation by endogenous processes in the nucleus of the target cell (Moore et al. 1993 The RNA World, eds. Gesteland, R. F. & Atkins, J. F. Cold Spring Harbor Lab. Press, Plainview, N.Y., pp. 303-357). Preferably, the two RNA strands that form the RNA duplex are held together by a spacer region that forms a loop. The spacer nucleotide sequence may be of any length. The order of the sense and antisense sequence is not essential, although an antisense-sense orientation is preferred because such a transcript is unlikely to code for a protein. It is also possible to combine more than one sense-antisense combination in one and the same construct. The simple form can be depicted as:

### prom-AS-spac-S-term

wherein prom represents a promoter, S the target viral DNA sequence, AS the target viral DNA sequence in opposite polarity compared to S, “spac” a spacer sequence and “term” the transcriptional terminator DNA sequence.

In addition, the following constructs may be used or applied:

### prom-AS1-spac-S1-spac-AS2-spac-S2-term

or

### prom-AS2-AS1-spac-S1-S2-term

Variations in the composition of the construct are possible, as long as the transcripts of said constructs may fold partially or fully into double stranded RNA of at least 50 base pairs in length.

By “promoter” is meant a sequence of nucleotides from which transcription may be initiated of DNA operably linked downstream (i.e. in the 3′ direction on the sense strand of double stranded DNA). “Operably linked” means joined as part of the same nucleic acid molecule, suitably positioned and oriented for transcription to be initiated from the promoter. DNA operably linked to a promoter is “under transcriptional initiation regulation” of the promoter.

The promoter may be a constitutive promoter, an inducible promoter or tissue-specific promoter. The terms “constitutive”, “inducible” and “tissue-specific” as applied to a promoter is well understood by those skilled in the art. The promoter is preferably derived from viruses, including 5′-long terminal repeats from retroviruses and lentiviruses, the SVEP, the CMVie and the like. Such promoters are readily available and are well known in the art.

By “terminator” is meant a sequence of nucleotides from which transcription may be terminated and a poly-A tail is added to the transcript. As terminator any terminator applicable in human or animal cells can be used.

Naturally, the skilled addressee will appreciate that the recombinant polyoma viral vectors, such as SV40 polyoma viral vectors, that are produced in the production cell lines of the invention will not comprise T antigen gene sequences and thus will not be capable of replication in a target cell.

Different polyoma viral vector production cell lines, eg SV40 viral vector production cell lines, are transfected with different DNA carrying vectors, such as plasmids, depending on their pedigree. The methodology for transfecting production cell lines is well known in the art. For example, if using the HEK293T cell line for the production of recombinant SV40 vectors for use in therapy and/or prophylaxis, HEK293T cells may be transfected with a first plasmid comprising the following components:

A promoter;

An RNA silencing suppressor sequence, for example that of E3L from vaccinia virus or other RNA silencing suppressor sequence;

A terminator; and optionally

A further promoter;

A selectable marker e.g. an antibiotic resistance marker such as hygromycin, puromycin, neomycin and the like; and

A further terminator.

Thus a single vector or plasmid could carry both an RNA silencing suppressor sequence and a selectable marker sequence. It is also possible that two separate DNA carrying vectors or plasmids could be utilised, one carrying the RNA silencing suppressor sequence and a second carrying the selectable marker, depending on design. Any further genetic elements that may be needed to confer a polyoma virus vector production capability on a production cell line may also be placed onto one or more DNA vectors or plasmids that may then be used to transfect the production cell line of choice. For instance, the VERO production cell line or the HEK293 production cell line does not already contain the polyoma virus T antigen sequences in it, so these should be added into it, and the resulting nascent production cells harbouring the T antigen sequences in them may then be selected for, by using a selectable marker system. Thus, in the example of utilising VERO or HEK293 cell lines, a suitable second further DNA-carrying vector or plasmid that may be used may contain the following components:

A heterologous promoter;

The nucleic acid sequence (DNA sequence) comprising the whole T antigen coding domain (stag, mtag and Tag sequences);

A heterologous terminator; and optionally

A further promoter;

A selectable marker eg an antibiotic resistance marker such as hygromycin, puromycin, neomycin and the like; and

A further terminator of choice.

As a third component that can be used to transfect a VERO cell line or a HEK293 cell line to produce SV40 virus vectors for use in anti viral therapy and/or prophylaxis, a third DNA vector comprising the following components can be employed:

A promoter such as SVEP comprising the SV40 ori;

An heterologous DNA sequence that is capable of being transcribed into an RNA sequence that is capable of folding into dsRNA of at least 50 base pairs in length, with nucleotide sequence homology to the target nucleic acid sequence of a vertebrate virus, such as the nucleic acid sequence of nef or the 5′-untranslated leader sequence of HIV-1 or the NS5 sequence or 5′-untranslated leader sequence of HCV or the sequence corresponding with the X gene of HBV; and a terminator. For example the native SV40 polyadenylation sequence may be used as a terminator.

Thus, as a preferred aspect of the invention there is provided an SV40 cell production cell line comprising

i) an RNA silencing suppressor sequence, for example that of E3L from vaccinia virus or other silencing suppressor sequence;

ii) a heterologous polynucleotide sequence with nucleotide sequence homology to the nucleic acid sequence of a vertebrate virus that is capable of being transcribed into an RNA sequence that is capable of folding into dsRNA of at least 50 base pairs in length.

An RNA silencing suppressor sequence is a polynucleotide sequence of a virus that when introduced in an eukaryotic cell encodes a protein or fragment thereof or RNA molecule, which is capable of interfering with RNA silencing and enhances/boosts and/or stabilises the expression of a nucleotide sequence encoding a (pharmaceutical) protein, a (therapeutic) monoclonal antibody, a virus or viral vector or an (industrial) enzyme and the like. Said protein or fragment thereof or RNA molecule comprises the non-structural protein (NSs) of the genus Tospovirus within the Bunyaviridae, the non-structural protein (NS1) of the Orthomyxoviridae, preferably that of influenza virus A, a non-structural protein VP35 of the Filoviridae, a non-structural protein E3L of the Poxviridae, preferably that of vaccinia virus, a non-structural protein (Tat of HIV-1/Tas of PFV-1) of the Retroviridae or the VA RNA molecules of the Adenoviridae.

The presence of the RNA silencing suppressor sequence acts to enhance the amounts or levels of polyoma viral vectors, such as, SV40 viral vectors, that are produced in the production cell lines of the invention, thus making such production cell lines comprising the RNA silencing suppressor sequence more efficient as SV40 viral vector “factories” than the conventional production cell lines of the prior art.

The heterologous polynucleotide sequence is one that is introduced into the SV40 cell production cell line via a suitable vector, such as a plasmid carrying DNA as herein described. The heterologous polynucleotide sequence is typically one that is made up of at least one inverted repeat of at least 50 base pairs in length corresponding to an essential sequence present in the target virus of interest, such as HIV-1 or hepatitis C virus or hepatitis B virus. For example, the essential sequence may be selected from the 5′-untranslated leader, tat, nef and rev coding domains of HIV-1, preferably the heterologous polynucleotide sequence of choice for use in constructing the recombinant polyoma virus is derived from the 5′-untranslated leader or nef coding domain of the HIV-1 subtype of interest, such as HIV-1 A, or is derived from the 5′-untranslated leader or ns5 coding domain of hepatitis C, or is derived from the sequence corresponding with the X gene of hepatitis B. The heterologous polynucleotide sequences of the invention are capable of being used in the prophylaxis and/or therapy of viruses that cause viral disease in humans, such as AIDS and viral hepatitis. Typically, the heterologous polynucleotide sequences of the invention are at least 50 base pairs in length. It is thought that the order of the actual orientation of the sense and antisense sequences of the heterologous polynucleotide of the invention is not material to the invention and the two portions may be presented either in the “sense-antisense” orientation, respectively, or the “antisense-sense” orientation, respectively. Preferably, the heterologous polynucleotide sequence of the invention is at least 50 base pairs in length, more preferably, about 100 base pairs in length, more preferably about 500 or more base pairs in length. For example, the heterologous polynucleotide of the invention may be 600, 700, 800, 900, 1000, 1100, 1200, 1300, 1400, 1500, 1600, 1700, 1800, 1900, 2000, 2100, 2200, 2300, 2400 or 2500 bases pairs in length.

In one aspect of the invention the heterologous polynucleotide sequence, for example dsDNA may be comprised of several sub-sequences of DNA that encode several different RNA sub sequences of different related or unrelated viruses. By way of illustration, HIV-1 has evolved into different sub-types that dominate different geographical locations. In Western

Europe and the Americas the subtype is predominantly B; in Africa, A and C; and in Eastern Europe A. For instance, one example of a heterologous DNA sequence may comprise the following components in the following order:

A promoter-an antisense sequence of HIV-1C-an antisense sequence of HIV-1B-an antisense sequence of HIV-1A-sense HIV-1A-sense HIV-1B-sense HIV-1C-a terminator.

In such a heterologous DNA sequence of the invention the sense and antisense sequences may be from 50-500 base pairs in length.

A similar example of a heterologous DNA sequence can be given for the different HCV sub-types.

Alternatively, an SV40 viral vector of the invention may be constructed such that the heterologous polynucleotide sequence of the invention can be used to inhibit the replication of two or more unrelated viruses that may be present in the same mammal host, such as a human. Such a component, for example, could have application in those cases where an individual has HIV-1 due to one strain of HIV-1 and has super-imposed on that condition a further disease caused by another virus such as hepatitis C. Such an heterologous polynucleotide sequence may be comprised as follows:

A promoter-antisense HIV-1 DNA sequence-antisense hepatitis C sequence-sense hepatitis C sequence-sense HIV-1 DNA sequence-terminator.

In such an heterologous DNA sequence of the invention, the individual viral sequences are at least 50 base pairs in length. Naturally, the skilled addressee will appreciate that the sense and antisense portions of the two above-described illustrations may be in a different order provided that when transcribed into RNA, they are capable of forming duplexes of at least 50 base pairs long that are recognised by DICER and that inhibit replication of the target virus or target viruses.

According to a further aspect of the invention there is provided use of the production cell line of the invention for the preparation of a pharmaceutical composition. Such a composition comprises a recombinant SV40 virus of the invention. The composition according to the invention may be used in the in vivo and/or ex vivo treatment of a subject in need who is suffering from a viral infection. The “subject in need” is typically a mammalian subject, such as a human patient who is suffering from a viral infection.

In further aspects of the invention there is provided a preparation of a pharmaceutical composition for the treatment of an individual suffering from a viral disease. The pharmaceutical composition may comprise a therapeutically effective amount of one or more, SV40 viruses of the invention prepared according to a process of the invention and a pharmaceutically acceptable carrier or diluent. The pharmaceutical compositions of the invention can be formulated in any suitable form for administration to the individual in need thereof. Such formulations may be in any form for administration such as topical, oral, parenteral, intranasal, intravenous, intramuscular, intralymphatic, subcutaneous, intraocular or even transdermal administration.

The pharmaceutical compositions of the invention generally comprise a buffering agent, an agent that adjusts the osmolarity thereof, an optionally, one or more pharmaceutically acceptable carriers, excipients and/or additives as known in the art. Supplementary active ingredients may also be incorporated into the compositions of the invention. The carrier may be a solvent or dispersion medium containing, for example, water, ethanol, polyol (for example, glycerol, propylene glycol, and liquid polyethylene glycol, and the like), suitable mixtures thereof, and vegetable oils. The correct fluidity may be maintained by the use of a coating, such as lecithin, by the maintenance of the required particle size in the case of dispersion and by the use of surfactants.

The invention will now be further described with reference to the following non-limiting Examples.

### EXAMPLE 1(a)

### Construction of the SV40 Vector (1)

DNA of a full-length SV40 DNA clone (ATCC number VRMC-2) was digested with EcoR1, the DNA fragment corresponding with SV40 was isolated from an agarose gel and circularized using T4 DNA ligase (Invitrogen), yielding SV40. SV40 was propagated by transfection of Vero cells as described under example 3. SV40 DNA was extracted from the SV40 virus stocks by phenol extraction and ethanol precipitation. Genomic SV40 DNA was digested with BamHI and Kpn1, and the resulting 2.2 kbp DNA fragment was isolated from an agarose gel and cloned into likewise digested pUC19 (Stratagene), yielding pUCSV40-BamH1-Kpn1. Four oligonucleotides were designed JvdV001: CCTCTGAAAGAGGAACTTGG (SEQ ID No.1), JvdV002: CAACAATTGCATT CATTGGCGCGCCGCGGCCGCTTAATTAACTTTGCAAAGCTTTTTGC (SEQ ID No.2), JvdV003: GCAAAAAGCTTTGCAAA GTTAATTAAGCGGCCGCGGCGCGCCAATGAATGCAATTGTTG (SEQ ID No.3) and JvdV004: CACAGAGGAGCTTCCTGG GGATCCGGTACCAG (SEQ ID No.4) (containing BamH1 and Kpn1 restriction sites). Purified genomic SV40 DNA was subjected to PCR using oligonucleotides JdV001 and JdV002 and oligonucleotides JvdV003 and JvdV004, yielding cDNA molecules comprising the SV40 promoter/ori region and the SV40 polyadenylation region, respectively. Both DNA fragments were agarose gel purified, mixed and subjected to a second round of PCR amplification using oligonucleotides JvdV001 and JvdV004. The resulting DNA fragment was digested with Kpn1, the 560 by long DNA fragment was isolated from an agarose gel and ligated into likewise digested pUCSV40-BamH1-Kpn1 DNA, yielding the recombinant plasmid pUCSV40-BamH1 with the Asc1 and Pad cloning sites.

### EXAMPLE 1(b)

### Construction of the SV40 Vector (2)

Six oligonucleotides are designed.

JvdV101:

CCGCTCGAGTTGCGGCCGCTGTGCCTTCTAGTTGCCAGCCATC (SEQ ID No.5) (containing a XhoI and a NotI restriction site) and

JvdV102: GGTACCATAGAGCCCACCGCATCCCCAGCATGCC (SEQ ID No.6) (containing a KpnI restriction site) and

JvdV103:

GGCCGCTTTATTAATTAAGCCCTGCAGGTTGTTTAAACTTGGCGC GCCTTAT (SEQ ID. No.7) (contains from 5′ to 3′ subsequently a NotI sticky restriction site, a PacI, SbfI, PmeI and an AscI intact restriction site and a ClaI sticky restriction site) and

JvdV104:

CGATAAGGCGCGCCAAGTTTAAACAACCTGCAGGGCTTAATTAAT AAAGC (SEQ ID No.8) (contains from 3′ to 5′ subsequently a NotI sticky restriction site, a PacI, SbfI, PmeI and an AscI intact restriction site and a ClaI sticky restriction site) and

JvdV105: CGGGATCCAGACATGATAAGATACATTG (SEQ ID No.9) (containing a BamHI restriction site) and

JvdV106: ATAGTTTAGCGGCCGCAACTTGTTTATTGCAGCTTATAATGG (SEQ ID No.10) (containing a NotI restriction site).

Purified plasmid DNA of the SV40 vector pSL-PL (de la Luna, S., Martin, J., Portela, A., and Ortin, J., 1993, Influenza virus naked RNA can be expressed upon transfection into cells co-expressing the three subunits of the polymerase and the nucleoprotein from simian virus 40 recombinant viruses. J. Gen. Virol. 74: 535-539) was subjected to PCR using oligonucleotides JvdV105 and JvdV106. The resulting amplified DNA fragment comprised the SV40-poly adenylation signal flanked by a BamHI restriction site on the 5′ end and a NotI restriction site on the 3′end. This SV40 poly adenylation signal fragment was digested with BamHI and NotI, and the resulting 150 by DNA fragment was isolated from an agarose gel and cloned into a likewise digested pBluescript SK− plasmid, yielding pHY290.

Purified pEF5/FRT/V5-DEST (Invitrogen) plasmid DNA was subjected to PCR using oligonucleotides JvdV101 and JvdV102.The resulting amplified DNA fragment comprised the BGH-poly adenylation signal flanked by subsequently a XhoI and a NotI restriction site on the 5′end and an KpnI restriction site on the 3′end. This BGH poly adenylation signal fragment was digested with KpnI and NotI, and the resulting 250 by DNA fragment was isolated from an agarose gel and ligated into the likewise digested pHY290 plasmid. Transformation with this ligation mixture was performed in a methylation insensitive E. coli strain. This resulted in plasmid pHY291.

The two complementary oligonucleotides JvdV103 and JvdV104 were annealed by incubating them in a water bath that was cooling down autonomously from boiling temperature to room temperature, yielding a DNA linker containing subsequently a NotI sticky restriction site, a PacI, SbfI, PmeI and a AscI intact restriction site and a ClaI sticky restriction site. This linker was ligated into the pHY291 plasmid that was digested with NotI and ClaI, and was isolated from agarose gel. The ligation mixture was subsequently used to transform a methylation insensitive E. coli strain, yielding pHY292.

Purified plasmid DNA of the SV40 vector pSL-PL was digested with ClaI and BamHI. The resulting 2.6 kb DNA fragment that contains the SV40 origin and the SV40 late region is purified from agarose and cloned into likewise digested pHY292. The resulted in the new SV40 vector plasmid pHY293.

### EXAMPLE 2(a)

### Construction of the Recombinant SV40 Vectors

Construction of a Recombinant SV40 Vector Comprising the HIV-1 Leader Sequence Followed By a Long Hairpin Nef Sequence of 300 bp Long.

Purified DNA of an infectious cDNA clone of HIV-1, denoted pLai (Peden K., et al., 1991. Virology 185:661-672) was used as a template, for cloning of the HIV-1 leader sequence and sequences corresponding with the Nef gene, using DNA-based PCR.

A DNA fragment comprising the HIV-1 leader was generated by PCR using pLai DNA as a template with oligonucleotides WdV001: CTTAATTAAGGGTCTCTCTGGTTAGACCAG (SEQ ID No.11) (containing a PacI restriction site) and WdV002: AGCGGCCGCAGTCGCC TCCCCTCGCCTCTTG (SEQ ID No.12) (containing a Not1 restriction site). The resulting DNA fragment was digested with Pac1 and Not1, isolated from an agarose gel and ligated in likewise digested pUCSV40-BamH1 DNA, yielding pUCSV40-BamH1-01.

A 320 base pairs anti-sense Nef DNA fragment was generated by PCR using pLai DNA as a template and oligonucleotides; WdV003: CAGGTGTCGTGAGTAGCACCATCCAAAGG (SEQ ID No.13) (containing a NotI restriction site) and WdV004: ATAGTTTAGCGGCCGCACAAG TAGCAATACAGCAGCTACC (SEQ ID No.14) (containing a Asc1 restriction site). The resulting DNA fragment was digested with Not1 and Asc1, isolated from an agarose gel and ligated in likewise digested pUCSV40-BamH1-01 DNA, yielding pUCSV40-BamH1-02.

A 300 base pairs sense Nef DNA fragment was generated by PCR using pLai DNA as a template and oligonucleotides; WdV005: AGCCTAGGACAAGTAGCAATACAGCAGCTAC (SEQ ID No.15) (containing an Asc1 restriction site) and WdV006: AGAAGGCAC AGGTAGCACCATCCAAAGGTC (SEQ ID No.16) (containing an Asc1 restriction site). The resulting DNA fragment was digested with Asc1, isolated from an agarose gel and ligated in likewise digested pUCSV40-BamH1-02 DNA. The plasmid with the Nef sequences as an inverted repeat was denoted pUCSV40-BamH1-03.

Construction of a Recombinant SV40 Vector Comprising a HIV-1 Long Hairpin Nef Sequence of 1000 bp Long.

A 1020 base pairs anti-sense Nef DNA fragment was generated by PCR using pLai DNA as a template and oligonucleotides; WdV007: GCTTAATTAACCAGCGGAAAGTCCCTTG (SEQ ID No. 17) (containing a Pac1 restriction site) and WdV008: GAGCGGCCGCC ACTTTGTACAAGAAAGC (SEQ ID No.12) (containing a Not1 restriction site). The resulting DNA fragment was digested with Pac1 and Not1, isolated from an agarose gel and ligated in likewise digested pUCSV40-BamH1 DNA, yielding pUCSV40-BamH1-04.

A 1000 base pairs sense Nef DNA fragment was generated by PCR using pLai DNA as a template and oligonucleotides; WdV009: GAGCGGCCGCACAGATCCATTCGATTAG (SEQ ID No.18) (containing a Not1 restriction site) and WdV010: GAGGCGCGCCAGCGGAAA GTCCCTTG (SEQ ID No. 19) (containing an Asc1 restriction site). The resulting DNA fragment was digested with Not1 and Asc1, isolated from an agarose gel and ligated in likewise digested pUCSV40-BamH1-04 DNA, yielding pUCSV40-BamH1-05.

Construction of a Recombinant SV40 Vector Comprising a HCV Long Hairpin NS5b Sequence of 1000 bp Long.

Purified DNA of an HCV 1b replicon clone, denoted Replicon-ET (Pietschmann T., et al., 2002. Journal of Virology 76: 4008-4021) was used as a template, for cloning of sequences corresponding with the NS5b cistron, using DNA-based PCR. A 1020 base pairs anti-sense NS5b DNA fragment was generated by PCR using Replicon-ET DNA as a template and oligonucleotides; WdV011: GCTTAATTAAGGTTGGGGAGTAGATAGATG (SEQ ID No.20) (containing a Pac1 restriction site) and WdV012: GAGCGGCCGCCGTGTTGAGGAGTCAATC (SEQ ID No.21) (containing a Not1 restriction site). The resulting DNA fragment was digested with Pac1 and Not1, isolated from an agarose gel and ligated in likewise digested pUCSV40-BamH1 DNA, yielding pUCSV40-BamH1-06.

A 1000 base pairs sense NS5b cistron DNA fragment was generated by PCR using Replicon-ET DNA as a template and oligonucleotides; WdV013: GAGCGGCCGCGAGCGGCTTTACATCGGG (SEQ ID No. 22) (containing an Not1 restriction site) and WdV014: GAGGCGCGCCA CTGTGCTGGATATCAAACC (SEQ ID No.23) (containing an Asc1 restriction site). The resulting DNA fragment was digested with Not1 and Asc1, isolated from an agarose gel and ligated in likewise digested pUCSV40-BamH1-06 DNA, yielding pUCSV40-BamH1-07.

### EXAMPLE 2(b)

### Construction of the Recombinant SV40 Vectors

Construction of a Recombinant SV40 Vector Comprising the HIV-1 Leader Sequence Followed By a Long Hairpin Nef Sequence of 300 bp Long.

Purified DNA of an infectious cDNA clone of HIV-1, denoted pLai (Peden K., et al., 1991. Virology 185:661-672) was used as a template, for cloning of the HIV-1 leader sequence and sequences corresponding with the Nef gene, using DNA-based PCR.

A DNA fragment comprising the HIV-1 leader was generated by PCR using pLai DNA as a template with oligonucleotides WdV101: CGGCGCGCCGGGTCTCTCTGGTTAGACCAG (SEQ ID No.24) (containing an Asc1 restriction site) and WdV102: AGTTTAAACAGTCGC CTCCCCTCGCCTCTTG (SEQ ID No.25) (containing a Pme1 restriction site). The resulting DNA fragment was digested with Asc1 and Pme1, isolated from an agarose gel and ligated in likewise digested pHY293 DNA, yielding pHY293-11.

A 320 base pairs anti-sense Nef DNA fragment was generated by PCR using pLai DNA as a template and oligonucleotides; WdV103: CGTTTAAACTGAGTAGCACCATCCAAAGG (SEQ ID No.26) (containing a Pme1 restriction site) and WdV104: GAGTTTAAACCACT TTGTACAAGAAAGC (SEQ ID. NO.27) (containing a Sbf1 restriction site). The resulting DNA fragment was digested with Pme1 and Sbf1, isolated from an agarose gel and ligated in likewise digested pHY293-11 DNA, yielding pHY293-12.

A 300 base pairs sense Nef DNA fragment was generated by PCR using pLai DNA as a template and oligonucleotides; WdV105: AGCCTGCAGGACAAGTAGCAATACAGCAGCTAC (SEQ ID No.28) (containing an Sbf1 restriction site) and WdV106: ATTAATTAA AGGTAGCACCATCCAAAGGTC (SEQ ID No.29) (containing an Pac1 restriction site). The resulting DNA fragment was digested with Sbf1 and Pact, isolated from an agarose gel and ligated in likewise digested pHY293-12 DNA, yielding pHY293-13.

Construction of a Recombinant SV40 Vector Comprising a HIV-1 Long Hairpin Nef Sequence of 1000 bp Long.

A 1020 base pairs anti-sense Nef DNA fragment was generated by PCR using pLai DNA as a template and oligonucleotides; WdV107: GCGGCGCGCCCCAGCGGAAAGTCCCTTG (SEQ ID No.30) (containing an Asc1 restriction site)and WdV108: GAGTTTAAACCACTTTG TACAAGAAAGC (SEQ ID No.31) (containing a Pme1 restriction site). The resulting DNA fragment was digested with Asc1 and Pme1, isolated from an agarose gel and ligated in likewise digested pHY293 DNA, yielding pHY293-14.

A 1000 base pairs sense Nef DNA fragment was generated by PCR using pLai DNA as a template and oligonucleotides; WdV109: GAGTTTAAACACAGATCCATTCGATTAG (SEQ ID No.31) (containing a Pme1 restriction site) and WdV110: GATTAATTAAAGCGGAAAGTCCCTTG (SEQ ID No.32) (containing a Pad restriction site). The resulting DNA fragment was digested with Pme1 and Pac1, isolated from an agarose gel and ligated in likewise digested pHY293-14 DNA, yielding pHY293-15.

Construction of a Recombinant SV40 Vector Comprising a HCV Long Hairpin NS5b Sequence of 1000 bp Long.

Purified DNA of an HCV 1b replicon clone, denoted Replicon-ET (Pietschmann T., et al., 2002. Journal of Virology 76: 4008-4021) was used as a template, for cloning of sequences corresponding with the NS5b cistron, using DNA-based PCR. A 1020 base pairs anti-sense NS5b DNA fragment was generated by PCR using Replicon-ET DNA as a template and oligonucleotides; WdV111: GCGGCGCGCCGGTTGGGGAGTAGATAGATG (SEQ ID No. 33) (containing an Asc1 restriction site) and WdV112: GAGTTTAAACCGTGTTGAGGAGTCAATC (SEQ ID No. 34) (containing a Pme1 restriction site). The resulting DNA fragment was digested with Asc1 and Pme1, isolated from an agarose gel and ligated in likewise digested pHY293 DNA, yielding pHY293-16.

A 1000 base pairs sense NS5b DNA fragment was generated by PCR using Replicon-ET DNA as a template and oligonucleotides; WdV113: GAGTTTAAACGAGCGGCTTTACATCGGG (SEQ ID No.35) (containing a Pme1 restriction site) and WdV014: GATTAATTAAACTGTGC TGGATATCAAACC (SEQ ID No. 36) (containing an Pac1 restriction site). The resulting DNA fragment was digested with Pme1 and Pac1, isolated from an agarose gel and ligated in likewise digested pHY293-16 DNA, yielding pHY293-17.

### EXAMPLE 3

### Construction of a Tag and RSS Expression Plasmid

An expression plasmid comprising the vaccinia virus E3L gene, the neomycin phospho transferase selectable marker gene and the SV40 Tag/tag genes was constructed by DNA-based fusion PCR.

An 1180 base pairs DNA fragment corresponding with the human EF-1alpha promoter was generated by PCR using pEF5/FRT/V5-Dest (Invitrogen) DNA as a template and oligonucleotides; JvdV005: GCGAATTCGTGAGGCTCCGGTGCCCG (SEQ ID No. 37) (containing an EcoR1 restriction site) and JvdV006: CAATATAGATCTTAGACAT GCACGACACCTGAAATGGA (SEQ ID No. 38). A 570 base pairs DNA fragment corresponding with the vaccinia virus E3L coding sequence was generated by PCR using purified vaccinia virus (strain Ankara) DNA as a template and oligonucleotides; JvdV007: TCCATTTCAGGTGTCGTGCAT GTCTAAGATCTATATTG (SEQ ID No. 39) and JvdV008: TGGCAACTAGAAGGCACAGCTAATGATGACGTAACCAAG (SEQ ID No. 22). A 220 base pairs DNA fragment corresponding with the human BGH terminator was generated by PCR using pEF5/FRT/V5-Dest (Invitrogen) DNA as a template and oligonucleotides; JvdV009: CTTGGTTACGTCATCATTAGCTGTGCCTTCTAGTTGCCA (SEQ ID No. 40) and JvdV010: GCGAATTCCATAGAGCCCCGCATCCCC (SEQ ID No. 24) (containing an EcoR1 restriction site). The 1180, 570 and 220 base pairs long DNA fragments were agarose gel purified, mixed and subjected to a second round of PCR amplification using oligonucleotides JvdV005 and JvdV010. The resulting DNA fragment was digested with EcoR1, the 1970 base pairs long DNA fragment was isolated from an agarose gel and ligated into likewise digested pBluescript-SK+ (Stratagene) DNA, yielding the recombinant plasmid pSK-E3L.

A 1480 base pairs DNA fragment corresponding with the TK promoter, the neomycin phosphotransferase coding sequence and the TK terminator was generated by PCR using pREP9 (Invitrogen) DNA as a template and oligonucleotides; JvdV011: GCGGATCCCCGGAAGAAATATATTTGC SEQ ID No. 41) (containing a BamH1 restriction site) and JvdV012: GCGGATCCGCTATGGCAGGGCCTGCCG (SEQ ID No. 42) (containing a BamH1 restriction site). The resulting DNA fragment was digested with BamH1 and ligated into likewise digested pSK-E3L DNA, yielding pSK-E3L-NEO.

An 1180 base pairs DNA fragment corresponding with the human EF-1alpha promoter was generated by PCR using pEF5/FRT/V5-Dest (Invitrogen) DNA as a template and oligonucleotides; JvdV013: GCGGTACCGTGAGGCTCCGGTGCCCG (SEQ ID No. 43) (containing a Kpn1 restriction site) and JvdV014: TGTTTAAAACTTTA TCCATGCACGACACCTGAAATGGA (SEQ ID No. 44). A 2470 base pairs DNA fragment corresponding with the SV40 Tag/tag coding sequence was generated by PCR using purified SV40 virus DNA as a template and oligonucleotides; JvdV015: TCCATTTCAGGTGTCGTGCATGGAT AAAGTTTTAAACA (SEQ ID No. 45) and JvdV016: TGGCAACTAGAAGGCACAGTTATGTTTCAGGTTCAGGGG (SEQ ID No.46). A 220 base pairs DNA fragment corresponding with the human BGH terminator was generated by PCR using pEF5/FRT/V5-Dest (Invitrogen) DNA as a template and oligonucleotides; JvdV017: CCCCTGAACCTGAAACATAACTGTGCCTTCTAGTTGCCA (SEQ ID No. 47)and JvdV0018: GCGGTACCATAGAGCCCCGCATCCCC (SEQ ID No.48) (containing an Kpn1 restriction site). The 1180, 2470 and 220 base pairs long DNA fragments were agarose gel purified, mixed and subjected to a second round of PCR amplification using oligonucleotides JvdV013 and JvdV018. The resulting DNA fragment was digested with Kpn1, the 3870 base pairs long DNA fragment was isolated from an agarose gel and ligated into likewise digested pSK-E3L-NEO DNA, yielding the recombinant plasmid pSK-E3L-NEO-TAG.

### EXAMPLE 4

### Generation of a Vero Producer Cell Line and Production of (Recombinant) SV40 Particles

African green monkey kidney Vero cells are grown at 37° C. in Dulbecco's modified Eagle medium with 0.11 grams per litre sodium pyridoxine, MEM non essential amino acids (Gibco), supplemented with 10% Fetal bovine serum (Biochrom KG), 100 units per millilitre penicillin and 100 μg per millilitre streptomycin (DME medium). Twice a week the confluent cell cultures are diluted 10 times in DME medium and sub cultured at 37° C.

pSK-E3L-NEO-TAG DNA was linearized by digestion with Not1.

Vero cells were transfected with linearized pSK-E3L-NEO-TAG DNA using the lipofectamin2000 method following the manufacturer's recommendations (Invitrogen) and the cells were grown and selected in appropriate medium provided with 100 μg/ml geneticin (Invitrogen). Cell clones were selected with pSK-E3L-NEO-TAG DNA stably integrated into the chromosomal DNA and further cultured in the presence of geneticin.

DNA of the (recombinant) SV40 vector plasmids pUCSV40-BamH1, pUCSV40-BamH1-03, pUCSV40-BamH1-05 and pUCSV40-BamH1-07 were digested with BamH1, the DNA fragments corresponding with the SV40 vector were isolated from agarose gels and circularized using T4 DNA ligase (Invitrogen), yielding SV40-EMPTY, SV40-03, SV40-05 and SV40-07 respectively.

Cell batches of individual neomycin-resistant clones were transfected with circularized DNA of the (recombinant) SV40 vectors using the lipofectamin2000 method, following the manufacturer's recommendations (Invitrogen). After transfection the cells were cultured for 3 days. Vector was harvested by three freeze-thaw cycles, followed by sonication. Cellular debris was removed by centrifugation at 2100 g for 30 minutes at 4° C. Vector stocks were prepared by three repeated infection cycles under identical conditions and the final virus titers were determined by in situ PCR as described by Vera M., et al., 2004, Molecular Therapy 10: 780-791. The best producer Vero cell clone was selected and subsequently used for the production of recombinant SV40 vector batches.

### EXAMPLE 5

### Generation of a HEK293 Producer Cell Line and Production of (Recombinant) SV40 Particles

HEK293 cells are grown at 37° C. in Dulbecco's modified Eagle medium with 0.11 grams per litre sodium pyridoxine, MEM non essential amino acids (Gibco), supplemented with 10% Fetal bovine serum (Biochrom KG), 100 units per millilitre penicillin and 100 μg per millilitre streptomycin (DME medium). Twice a week the confluent cell cultures are diluted 10 times in DME medium and sub cultured at 37° C.

pSK-E3L-NEO-TAG DNA was linearized by digestion with Not1.

HEK293 cells were transfected with linearized pSK-E3L-NEO-TAG DNA using the lipofectamin2000 method, following the manufacturer's recommendations (Invitrogen) and the cells were grown and selected in appropriate medium provided with 100 μg per milllitre geneticin (Invitrogen). Cell clones were selected with pSK-E3L-NEO-TAG DNA stably integrated into the chromosomal DNA and further cultured in the presence of geneticin.

DNA of the (recombinant) SV40 vector plasmids pHY293, pHY293-13, pHY293-15 and pHY293-17 were digested with Not1, the DNA fragments corresponding with the SV40 vector were isolated from agarose gels and circularized using T4 DNA ligase (Invitrogen), yielding recombinant vector SV40-10, SV40-13, SV40-15 and SV40-17 respectively.

Cell batches of individual neomycin-resistant clones were transfected with circularized DNA of the (recombinant) SV40 vectors using the lipofectamin2000 method, following the manufacturer's recommendations (Invitrogen). After transfection the cells were cultured for 3 days. Vector was harvested by three freeze-thaw cycles, followed by sonication. Cellular debris was removed by centrifugation at 2100 g for 30 minutes at 4° C. Vector stocks were prepared by three repeated infection cycles under identical conditions and the final virus titers were determined by quantitative PCR as described by Vera M., et al., 2004, Molecular Therapy 10: 780-791. The best producer HEK293 cell clone was selected and subsequently used for the production of recombinant SV40 vector batches.

### EXAMPLE 6

### SupT1 Cells Transduced with SV40-13 and SV40-15 are Resistant to HIV-1 Replication

SupT1 human non-Hodgkin's T-lymphoma cells (Smith S. D., et al., 1984. Cancer Research 44: 5657) are grown at 37° C. in RPMI 1640 medium with 2 mmol/L L-Glutamine (Gibco) supplemented with 10% Fetal bovine serum (Biochrom KG), 100 U/ml penicillin and 100 μg/ml streptomycin. Twice a week the cell cultures containing 2 times 10⁶ cells/ml are diluted 10 times in RPMI medium and sub cultured at 37° C.

10⁸ transducing units of recombinant SV40 vector was added to three ml of a SupT1 cell culture containing 10⁶ cells/ml and incubated for 2 hours at 37° C. Three days post transduction the SupT1 cell cultures are inoculated with 0.2 μl of SupT1-adapted Lai and the syncytium formation is monitored daily up to 14 days post infection. Syncytia are formed 6 days post infection in the cultures transduced with the control plasmids: SV40-10 and SV40-17. The numbers of syncytia formed in the cell cultures transduced with SV40-13 and SV40-15 are much lower.

### EXAMPLE 7

### Huh7 Cells Transduced with SV40-17 are Resistant to HCV Replication

Huh7 human hepatoma cells are grown at 37° C. in Dulbecco's Modified Eagle (DME) medium (Gibco) supplemented with 10% Fetal bovine serum (Biochrom KG), 100 U/ml penicillin and 100 μm μg/ml streptomycin. Twice a week the confluent cell cultures are diluted 10 times in DME medium and sub cultured at 37° C.

10⁸ transducing units of recombinant SV40 vector was added to three ml of a Huh7 cell culture containing 10⁶ cells/ml and incubated for 2 hours at 37° C. Three days post transduction the Huh7 cell cultures are transfected with HCV replicon RNA derived from Replicon-ET (Pietschmann T., et al., 2002. Journal of Virology 76: 4008-4021) as described by Lohmann V., et al., 2003, Journal of Virology 77: 3007-3019. The cells were maintained for 1 week and 105 cells were plated in 100 mm2 dishes and maintained in DME medium in the presence or absence of 0.75 mg/ml geneticin for 10 days. The cells were then washed, stained and counted as described by Randall G., et al., 2003, Proc. Natl. Acad. Sci USA, 100: 235-240. The number of geneticin-resistant cells transduced with SV40-17 is significantly higher than those transduced with the control vector SV40-10, SV40-13 and SV40-15.

1. A polyoma viral vector production cell line comprising a heterologous polynucleotide sequence that is capable of being transcribed into an RNA sequence that is capable of folding into double stranded RNA of at least 50 base pairs in length, said RNA polynucleotide sequence having substantial homology to a target nucleic acid sequence of a vertebrate virus, the target nucleic acid sequence being an essential sequence of the said virus.

2. A polyoma viral vector production cell line according to claim 1 wherein the heterologous polynucleotide sequence is selected from the 5′-untranslated leader, tat, nef and/or rev coding domains of HIV-1, the 5′-untranslated leader or ns5 coding domain of hepatitis C, or the X gene of hepatitis B.

3. A polyoma viral vector production cell line according to claim 1 wherein the heterologous polynucleotide sequence is selected from the 5′-untranslated leader or nef coding domain of an HIV-1 subtype.

4. A polyoma viral vector production cell line according to claim 1, wherein the heterologous polynucleotide sequence is at least 100 base pairs in length.

5. A polyoma viral vector production cell line according to claim 1, wherein the heterologous polynucleotide sequence is at least 500 base pairs in length.

6. A polyoma viral vector production cell line according to claim 1, wherein the heterologous polynucleotide sequence is at least 600 base pairs in length.

7. A polyoma viral vector production cell line according to claim 1, wherein the heterologous polynucleotide sequence is at least 700 base pairs in length.

8. A polyoma viral vector production cell line according to claim 1, wherein the heterologous polynucleotide sequence is at least 800 base pairs in length.

9. A polyoma viral vector production cell line according to claim 1, wherein the length of the said heterologous polynucleotide sequence is from 50 base pairs up to 2500 base pairs in length.

10. A polyoma viral vector production cell line according to claim 1, further comprising a second heterologous polynucleotide sequence that encodes a compatible viral nucleic acid silencing suppressor protein.

11. A polyoma viral vector production cell line according to claim 1, further comprising a second heterologous polynucleotide sequence that encodes a compatible viral RNA silencing suppressor sequence.

12. A polyoma viral vector production cell line according to claim 1, further comprising a second heterologous polynucleotide sequence that encodes a compatible viral nucleic acid silencing suppressor sequence selected from the (NSs) of the genus Tospovirus within the Bunyaviridae, the non-structural protein (NS1) of the Orthomyxoviridae, preferably that of influenza virus A, the non-structural protein VP35 of the Filoviridae, the non-structural protein E3L of the Poxviridae, preferably that of vaccinia virus, the non-structural protein (Tat/Tas) of the Retroviridae, preferably Tas of PFV-1, or the VA RNA molecules of the Adenoviridae.

13. A recombinant polyoma viral vector comprising:
i) a promoter;
ii) at least one antisense sequence of a target sequence of at least one virus or virus;
iii) at least one sense sequence to the said target sequence of ii); and
iv) a terminator.

14. A recombinant polyoma viral vector according to claim 13 comprising:
i) a promoter;
ii) at least two antisense sequences of two target sequences of at least two viruses or virus subtypes;
iii) at least two sense sequences to the said target sequences of ii); and
iv) a terminator.

15. A recombinant polyoma viral vector according to claim 13 comprising:
i) a promoter;
ii) a first antisense sequence of a target sequence of a first virus or virus subtype;
iii) a second antisense sequence of a target sequence of a second virus or virus subtype;
iv) a third antisense sequence of a target sequence of a third virus or virus subtype;
v) a sense sequence to the said third antisense sequence of the said third virus or virus subtype;
vi) a sense sequence of the said second antisense sequence of the said second virus or second virus subtype;
vii) a sense sequence of the said first antisense sequence of the said first virus or first virus subtype; and
viii) a terminator.

16. A recombinant polyoma viral vector according to claim 13 wherein the polyoma viral vector is an SV40 viral vector.

17. A recombinant polyoma viral vector according to claim 13 wherein the heterologous polynucleotide sequence is selected from the 5′-untranslated leader, tat, nef and/or rev coding domains of HIV-1, the 5′-untranslated leader or ns5 coding domain of hepatitis C, or the X gene of hepatitis B.

18. A recombinant polyoma viral vector according to claim 13 further comprises a further heterologous polynucleotide sequence that encodes a compatible viral nucleic acid silencing suppressor sequence selected from the (NSs) from a tospovirus, the non-structural protein (NS 1) of an influenza virus A, the non-structural protein VP35 of the Filoviridae, the non-structural protein E3L of a pox virus, preferably that of vaccinia virus, the non-structural protein (Tat/Tas) of the Retroviridae, preferably Tas of PFV-1, and the VA RNA molecule of an adenovirus.

19. A host cell that comprises the said heterologous polynucleotide sequence of the vector of claim 13 inserted therein.

20. A method of producing a recombinant SV40 vector according to claim 13 that comprises:
1. deleting Tag genes from a wild type SV40 vector;
2. inserting a first polylinker in front of the SV40 early promoter;
3. optionally introducing a second polylinker into the first polylinker;
4. inserting at least a first heterologous polynucleotide sequence according to any one of claims 13 to 18 into the said first polylinker and/or optionally added second polylinker;
5. circularising the vector;
6. introducing the circularised vector into a production cell line.

21. A method according to claim 20 wherein the production cell line is a VERO cell line, a 293 cell line or a 293T cell line.

22. An isolated polynucleotide sequence that encodes at least one heterologous polynucleotide sequence that is capable of being transcribed into an RNA sequence that is capable of folding into double stranded RNA of at least 50 base pairs in length, said polynucleotide sequence having substantial homology to a target nucleic acid sequence of a vertebrate virus, the target nucleic acid sequence being an essential sequence of the said virus.

23. An isolated polynucleotide sequence according to claim 22 wherein the heterologous polynucleotide sequence is at least 100 base pairs in length.

24. An isolated polynucleotide sequence according to claim 22 wherein the heterologous polynucleotide sequence is at least 500 base pairs in length.

25. An isolated polynucleotide sequence according to claim 22 wherein the heterologous polynucleotide sequence is at least 600 base pairs in length.

26. An isolated polynucleotide sequence according to claim 22 wherein the heterologous polynucleotide sequence is at least 700 base pairs in length.

27. An isolated polynucleotide sequence according to claim 22 wherein the heterologous polynucleotide sequence is at least 800 base pairs in length.

28. An isolated polynucleotide sequence according to claim 22 wherein the heterologous polynucleotide sequence is from 50 base pairs up to 2500 base pairs in length.

29. A method of producing a host cell according to claim 19, the method including incorporating a polynucleotide or nucleic acid vector into the cell by means of transfection.

30. Use of a polynucleotide according to claim 22 in the production of a polyoma viral vector production cell line.

31. Use of a polynucleotide according to claim 30 in the production of a polyoma viral vector production cell line that is an SV40 viral vector production cell line.

32. Use of a polyoma viral vector production cell line according to claim 1 for the preparation of a pharmaceutical composition.

33. A polyoma viral vector production cell line according to claim 1 that harbours an SV40 viral vector comprising i) wild type T antigen genes; and ii) an RNA Silencing Suppressor sequence.

34. A polyoma viral vector production cell line according to claim 33 that comprises the Tag gene.

35. A polyoma viral production cell line according to claim 33 which is an HEK 293 cell line.
